(19) United States
(12) Patent Application Publication
(10) Pub. No.: US 2021/0295262 A1
(43) Pub. Date: Sep. 23, 2021

(54) Patient Outcome-Based Data Store

(71) Applicant: Universal Research Solutions, LLC, Columbia, MO (US)

(72) Inventor: Ali Adel Hussam, Columbia, MO (US)

(21) Appl. No.: 17/086,233

(22) Filed: Oct. 30, 2020

Related U.S. Application Data
(63) Continuation of application No. 13/046,028, filed on Mar. 11, 2011, now abandoned.
Application No. 13/046,028 is a continuation-in-part of application No. 12/699,522, filed on Feb. 3, 2010, now Pat. No. 8,429,547.
(60) Provisional application No. 61/253,398, filed on Oct. 20, 2009.
(60) Provisional application No. 61/389,004, filed on Oct. 1, 2010.

(51) Int. Cl. G06Q 10/10; G16H 70/00

(52) U.S. Cl. CPC G06Q 10/10; G16H 70/00

(57) ABSTRACT

A computer-implemented includes receiving, by one or more computers, medical data that is a candidate for purchase in a data store; sending the received medical data to a regulatory engine for review; receiving, from the regulatory engine, approval to publish the medical data in the data store; and publishing the medical data in the data store.

### CLAIM OF PRIORITY

This application is a continuation-in-part and claims priority under 35 U.S.C. § 120 to U.S. patent application Ser. No. 12/699,522, filed on Feb. 3, 2010, which in turn claims priority under 35 U.S.C. § 119(e) to provisional U.S. Patent Application 61/253,398, filed on Oct. 20, 2009, the entire contents of each of which are hereby incorporated by reference. This application also claims priority under 35 U.S.C. § 119(e) to provisional U.S. Patent Application 61/389,004, filed on Oct. 1, 2010, the entire contents of which are also incorporated herein by reference.

### BACKGROUND

Medical forms are used to collect data and information regarding a patient's symptoms and conditions. One technique for preparing a medical form is to manually edit a pre-existing form (e.g., a form existing in Microsoft Word™ format) with new or customized questions. The form is then sent to review boards for review through a physical or electronic mailing. Additionally, once a form has been finalized, it may be presented to a patient, study participant or other individual (collectively referred to as “patients” herein, without limitation, for purposes of convenience). For example, physicians may present patients with the forms when the patient visits the physician's office. Additionally, hardcopy (i.e., paper) versions of medical forms may be distributed to patients for completion. For patient's who have not completed medical forms prior to the patient's examination, the patient may often complete the medical form at the physician's office by filling out a hardcopy of the form.

Frequently, the patient's responses to the questions included in the medical forms are entered into a computerized system by medical personnel. In this case, in order for a physician to review the patient's responses, the physician may access the computerized system and view the answers to the questions, which is often a lengthy process of reviewing individual questions.

### SUMMARY

In one aspect of the present disclosure, a computer-implemented method includes receiving, by one or more computers, medical data that is a candidate for purchase in a data store; sending the received medical data to a regulatory engine for review; receiving, from the regulatory engine, approval to publish the medical data in the data store; and publishing the medical data in the data store.

Implementations of the disclosure may include one or more of the following features. In some implementations, the data store includes a graphical user interface that when rendered on a display device renders a visual representation through which a consumer may view medical data. The method may also include receiving from a user a request to purchase medical data from the data store; verifying that an access level of the user matches an access level associated with the medical data; and sending to the user the purchased medical data.

In other implementations, the method includes verifying that the medical data may be formatted in accordance with one or more data format specifications; and generating a package from the medical data, with the package including the medical data formatted according to the one or more data format specifications. In still other implementations, the method includes generating a graphical user interface that when rendered in a display device renders a visual representation of at least some of the medical data included in the package. The method may also include receiving, by the one or more computers, a transaction fee for the requested medical data.

In another aspect of the disclosure, one or more machine-readable media are configured to store instructions that are executable by one or more processing devices to perform operations including receiving medical data that is a candidate for purchase in a data store; sending the received medical data to a regulatory engine for review; receiving, from the regulatory engine, approval to publish the medical data in the data store; and publishing the medical data in the data store. Implementations of this aspect of the present disclosure can include one or more of the foregoing features.

In still another aspect of the disclosure, an electronic system includes one or more processing devices; and one or more machine-readable media configured to store instructions that are executable by the one or more processing devices to perform operations including: receiving medical data that is a candidate for purchase in a data store; sending the received medical data to a regulatory engine for review; receiving, from the regulatory engine, approval to publish the medical data in the data store; and publishing the medical data in the data store. Implementations of this aspect of the present disclosure can include one or more of the foregoing features.

All or part of the foregoing may be implemented as a computer program product including instructions that are stored on one or more non-transitory machine-readable storage media, and that are executable on one or more processing devices. All or part of the foregoing may be implemented as an apparatus, method, or electronic system that may include one or more processing devices and memory to store executable instructions to implement the stated functions.

The details of one or more implementations are set forth in the accompanying drawings and the description below. Other features, objects, and advantages will be apparent from the description and drawings, and from the claims.

### BRIEF DESCRIPTION OF THE FIGURES

FIG. 1 is a conceptual diagram of a system that generates a patient-outcome based data store.

FIG. 2 is a block diagram of components of the system that generates the patient-outcome based data store.

FIG. 3 is a flow chart of generating a package for a data store.

FIG. 4 is a flow chart of a process for selling a package in the data store.

Like reference symbols in the various drawings indicate like elements.

### DETAILED DESCRIPTION

Described herein is a system for generating a data store through which medical data may be accessed. In an exemplary embodiment described herein, the medical data is collected from a medical study, as described in U.S. Ser. No. 12/699,522. In another exemplary embodiment described herein, the medical data includes patient outcome-based data. Generally, patient outcome-based data includes data collected by the system to evaluate the capacity of a medical procedure and/or process to function at a pre-defined level.

In an exemplary embodiment described herein, the medical data is collected and submitted to the system by physicians, nurses, health care providers, experts, researchers, reviewers, research members, clinic members and other individuals (collectively referred to as “data owners” herein, without limitation, for purposes of convenience). The medical data is accessed by individuals looking to utilize medical data, including, e.g., researchers, vendors, government entities, and so forth (collectively referred to as “data seekers” herein, without limitation, for purposes of convenience). Generally, data seekers include researchers, vendors, government entities, and so forth. Prior to being accessible to data seekers, the medical data is processed through a regulation portal to de-identify the medical data. In this exemplary embodiment, the medical data is de-identified by removing from the medical data information that identifies an individual (e.g., a patient) associated with the medical data. In another example, the medical data that is provided to the data seeker includes identifying information. Medical data submitted to the system is regulated based on standards of the American Medical Association (“AMA”) and other known standards to enforce ethics, privacy, and so forth.

In another exemplary embodiment described herein, the system includes a data repository that is configured to store the submitted medical data. The system is also configured to mine data from the data repository, for example, using the Data Mining and Research Tools Module described in U.S. Ser. No. 12/699,522. Additionally, the system is further configured to tag the submitted medical data with an identifier specifying a category of the submitted medical data. By associating a category with submitted medical data, the system is able to distinguish between various types of submitted medical data.

In an exemplary embodiment described herein, the system is configured to generate an access level for submitted medical data. Generally, an access level specifies a type of data seeker that may access the medical data. The types of access levels include subscription access levels, one time purchase access levels, and public access levels. In this exemplary embodiment, the system may be configured to store medical data based on the access level of the medical data. In another exemplary embodiment, the system includes data mining techniques for mining data based on the access level of the data.

In another exemplary embodiment described herein, a public access level includes a level of access in which medical data is generally available. Medical data associated with a public access level includes de-identified data that may be mined to generate reports, charts, statistical data, etc. When a data seeker accesses data associated with a public access level, the data seeker may view and/or download the medical data. Additionally, data seekers that have public access may also request direct communication with data owners, for example, if a data seeker has a question about a specific set of medical data.

A subscription access level provides access to the system to data seekers that have purchased a subscription to the system. In an exemplary embodiment, medical data that is associated with an access level of subscription data seeker is only accessible by data seekers that have subscribed to the system. Data seekers can subscribe to a pool of studies based on a subscription fee that will be approved by the data owner and a regulatory board. In an exemplary embodiment, a subscription can be annual/monthly. Data may be permitted to be downloaded. Data can be identified or de-identified. In this exemplary embodiment, a more restrictive and secure hosting environment will be imposed for the identified data option. A fee can be collected by the system described herein and passed to the data owner after deducting a handling fee. In an exemplary embodiment, a subscription level access may also provide the data seeker with additional functionality, including, e.g., the ability to post requests for studies, additional data mining tools and so forth.

Additionally, there may be varying types of subscription level access, including, e.g., subscription level access for a researcher, for a vendor, and for the government. In subscription level access for a researcher, a researcher will be able to access research related data stores. In subscription level access for a vendor, a vendor will be able to access medical instrument driven studies and data. In subscription level access for a government entity, a government user will have access to Food and Drug Administration (“FDA”) studies and government related studies. A one time purchase access level provides access to medical data that may be purchased, for example, in a one-time point of sale transaction by a data seeker.

In another exemplary embodiment, based on the access level of the medical data, the system is configured to determine if a portion of the medical data may be released to the general public, for example, to be previewed by the general public. In this exemplary embodiment, the system may generate a description of medical data and/or an abstract of the medical data that may be viewed in the data store by data seekers. In an exemplary embodiment, the system may generate a description of medical data that is associated with a one-time purchase access level. In this exemplary embodiment, the description is designed to provide a data seeker with information that may assist the data seeker in deciding whether to purchase the medical data.

In another exemplary embodiment, a data seeker may access the data store and request to purchase medical data satisfying certain criteria. In this exemplary embodiment, the system may not include medical data satisfying the criteria. The data seeker may post a request to the data store to seek a data owner to generate and collect the requested medical data.

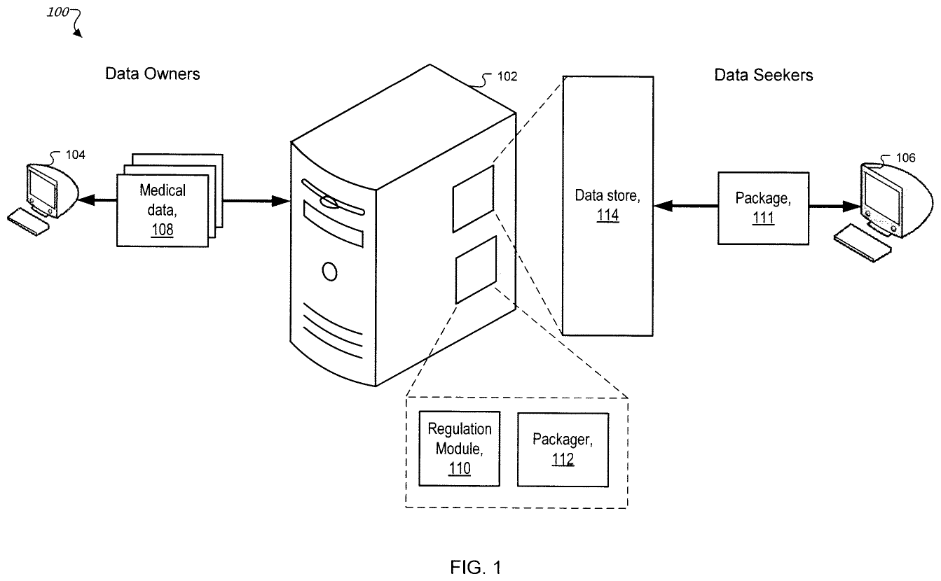

FIG. 1 illustrates a particular exemplary embodiment describe herein. In particular, FIG. 1 is a conceptual diagram of system 100 that generates data store 114 for the purchase of medical data. In the exemplary embodiment of FIG. 1, system 100 includes server 102 an client devices 104, 106. Client device 104 includes a computing device that is used by a data owner. Client device 106 includes a computing device that is used a data seeker.

In the exemplary embodiment of FIG. 1, a data owner uses client device 104 to submit medical data 108 to server 102. Server 102 includes numerous components, including, e.g., regulation module 110, packager 112 and data store 114. Regulation module 110 is configured to send medical data 108 to a regulatory board to approve medical data 108 for access by data owners via data store 114. Packager 112 is configured to generate package 111 that includes medical data 108 that has been formatted to be accessible through data store 114. In an exemplary embodiment, data store 114 includes an interface through which data seekers may access and/or view medical data 108. In an exemplary embodiment, data store 114 includes a graphical user interface that may be displayed on a computing device, including, e.g., client device 106. The data seeker using client device 106 may access package 111 through data store 114. In the exemplary embodiment of FIG. 1, package 111 is sent to client device 106.

In an exemplary embodiment described herein, packager 112 is further configured to organize packages to promote searching of medical data based on terms included in medical data, based on statistical information obtained from medical data 108, and so forth. In an exemplary embodiment, medical data submitted by a doctor be associated with many patients. In this exemplary embodiment, each patient is associated with multiple questionnaires. System 100 is configured to index and/or to store the medical data such that the medical data is searchable by the name of the doctor, the name of a patient, a name of a questionnaire, an outcome of a questionnaire, and so forth.

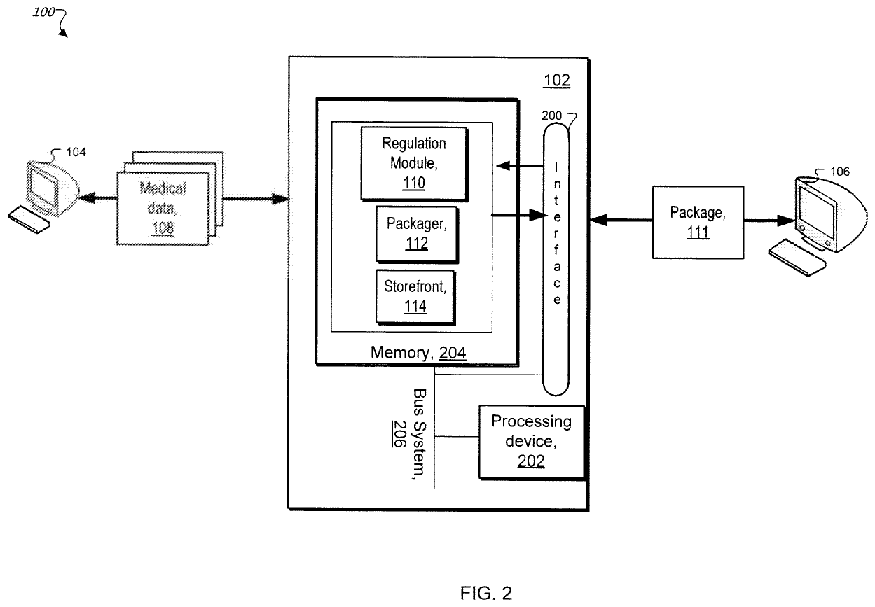

FIG. 2 illustrates a particular exemplary embodiment describe herein. FIG. 2 is a block diagram of components of system 100 generates data store 114 for the purchase of medical data. Client devices 104, 106 can be any sort of computing devices capable of taking input from a user and communicating over a network (not shown) with server 102 and/or with other client devices. For example, client devices 104, 106 can be mobile devices, desktop computers, laptops, cell phones, personal digital assistants (“PDAs”), servers, embedded computing systems, and so forth.

In the exemplary embodiment of FIG. 2, server 102 can be any of a variety of computing devices capable of receiving information, such as a server, a distributed computing system, a desktop computer, a laptop, a cell phone, a rack-mounted server, and so forth. Server 102 may be a single server or a group of servers that are at a same location or at different locations.

The illustrated server 102 can receive information from client device 104 via input/output (“I/O”) interface 200. I/O interface 200 can be any type of interface capable of receiving information over a network, such as an Ethernet interface, a wireless networking interface, a fiber-optic networking interface, a modem, and so forth. Server 102 also includes a processing device 202 and memory 204. A bus system 206, including, for example, a data bus and a motherboard, can be used to establish and to control data communication between the components of server 102.

The illustrated processing device 202 may include one or more microprocessors. Generally, processing device 202 may include any appropriate processor and/or logic that is capable of receiving and storing data, and of communicating over a network (not shown). Memory 204 can include a hard drive and a random access memory storage device, such as a dynamic random access memory, or other types of non-transitory machine-readable storage devices. As shown in FIG. 2, memory 204 stores computer programs that are executable by processing device 202. Among these computer programs are regulation module 110, packager 112, and data store 114.

In an exemplary embodiment, regulation module 110 is configured to promote an internal regulatory board for packages generated by system 100 and for medical data 108 received by system 100. In this exemplary embodiment, regulation module 110 provides a forum in which members of a regulatory board can review submitted medical data and generated packages, for example, before the packages are accessible in data store 114. In this exemplary embodiment, regulation module 110 enables the regulatory board to have access to secure threaded discussions to review, rate, vote and approve/disapprove submitted requests to have medical data generated into a package. Regulation module 110 is also configured to receive comments from data owners. Using the received comments, regulation module 110 categorizes a package as having subscription level access, one-time purchase access, or public access. In an exemplary embodiment, regulation model 110 is further configured to post the package to data store 114, for example, based on the access level associated with the package. In another exemplary embodiment, regulation module 110 is further configured to generate an alert that notifies the regulatory board of submitted package for review, for example, to approve or disapprove submission the package to data store 114.

In an exemplary embodiment, packager 112 is configured to format medical data 108 to preserve the integrity of medical data 108 in package 111. In this exemplary embodiment, package 111 includes information indicative of patient information and study information. Patient information includes information associated with the patient for which the medical data was submitted, including, e.g., demographic information, name information, and so forth. Study information includes information associated with a study in which medical data 108 was collected, including, e.g., study name, study description, the dates for which the study was run, study type (e.g., government funded, vendor specific, research specific, etc.), inclusive/exclusive criteria (e.g., medical codes, physical requirements such as height or weight, and any other miscellaneous criteria such as patients having diabetes or smoking habits), instruments used (e.g., a list of instruments/questionnaires used to determine the outcomes of the patients within the study), consent forms (e.g., verification and copies of the patient acceptance of the consent forms used), internal regulatory board approval (e.g., copies of approval by an internal regulatory board for the study), and so forth.

Data store 114 is configured to provide a visual representation of packages that have been approved by the regulatory body. In an exemplary embodiment, data store 114 includes a graphical user interface that when displayed in a display renders the visual representation of the packages. In an exemplary embodiment, by accessing data store 114, a data seeker may view, access, and/or purchase a package. In an exemplary embodiment, package 111 is cleansed prior to be being displayed in data store 114. Generally, cleansing includes removing certain predefined words and terms or removing certain information that identifies a patient.

Server 102 may also be configured to generate a data store for submission of proposals, for example, from data seekers. In this exemplary embodiment, the data store for submission of proposals will provide a portal for data providers and data seekers to collaborate on perspective studies. In an exemplary embodiment, collaboration agreements can be mediated through the portal. Additionally, data owners will be able to search for study proposals that are being offered by data seekers. Through the data store, a data owner may contact a data seeker to proceed with the proposal. Additionally, through the data store, data seekers will be able to post study proposals that can be searched for by data owners.

In an exemplary embodiment, regulation module 110 may also be configured to notify the regulatory board of a submitted proposal. The regulatory board may review the submitted proposal for compliance with certain pre-defined criteria, including, e.g., ethical use criteria and price point set criteria. Following an approval of the proposal by the regulatory board, the proposal is sent to the data store for access by data owners. In an exemplary embodiment, the proposal may be cleaned prior to being sent to the data store 114.

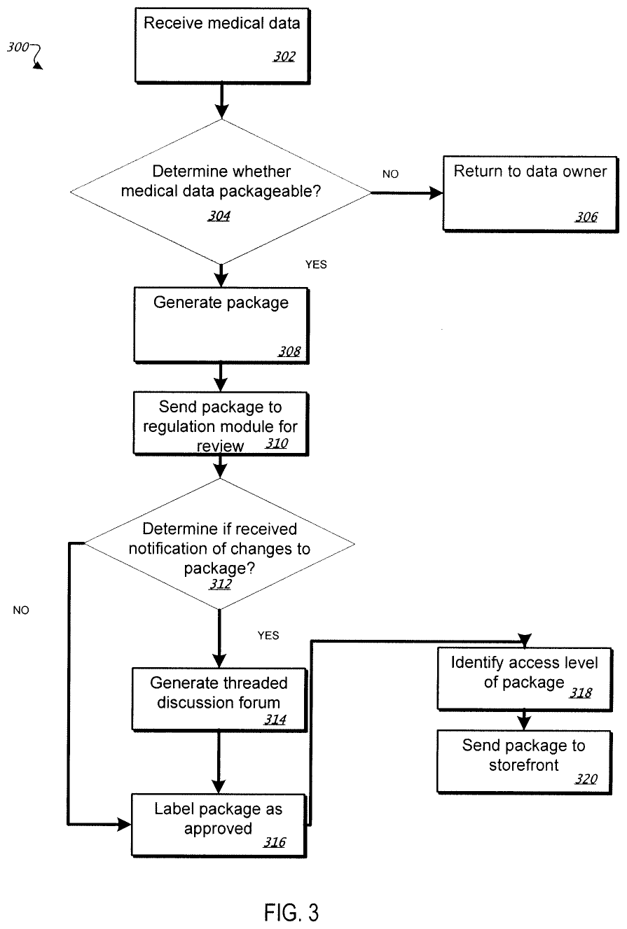

FIG. 3 illustrates a particular exemplary embodiment describe herein. In particular, FIG. 3 is a flow chart of process 300 for making package 111 accessible through data store 114. In operation, server 102 receives (302) medical data 108, for example, from client device 104 associated with a data owner. In an exemplary embodiment, the data owner submits to server 102 medical data 108 that the data owner wishes to be sold in data store 114.

Packager 112 determines (304) whether medical data 108 is packageable. Generally, packageable refers to a data format that complies with predetermined format specifications. If packager 112 determines that medical data 108 is not packageable, medical data 108 is returned (306) to the data owner. If packager 112 determines that medical data 108 is packageable, packager generates (308) package 111 including medical data 108. Packager 112 sends (310) package 111 to regulation module 110 so that the regulatory board may review package 111.

In an exemplary embodiment described herein, the regulatory board reviews package 111 to determine if package 111 complies with certain pre-defined criteria, including, e.g., Health Insurance Portability and Accountability Act (“HIPPA”) clearance criteria, ethical user criteria, data format criteria, quantity criteria, price point criteria, and so forth. If the regulatory board recommends that changes be made to package 111 prior to package 111 being made accessible in data store 114, the regulatory board submits to regulation module 110 a notification of requested changes. In an exemplary embodiment, regulation module 110 determines (312) if it has received a notification of changes to package 111. If regulation module 110 has received a notification of changes to package 111, regulation module 110 generates (314) a threaded discussion forum in which changes to package 111 may be discussed among the members of the regulatory board.

In the illustrative example of FIG. 3, following a determination by regulation module 110 that there are no additional notifications of changes to package 111, regulation module 110 labels (316) package 111 as approved. Regulation module 110 additionally identifies (318) an access level for package 111, for example based on input from the data owner of package 111 as previously described. Regulation module 110 sends (320) package 111 to data store 114 for access by data seekers. In an example, the package is sent to data store 114 by being published to data store 114. Generally, publishing includes making data accessible to users of the system, including, e.g., for purchase from the system.

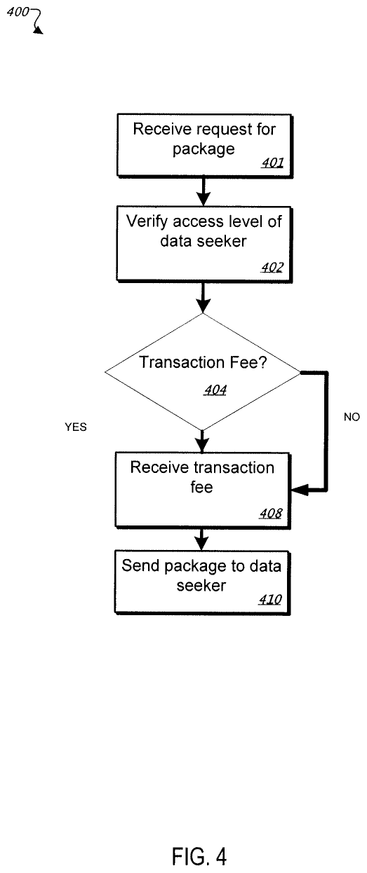

FIG. 4 illustrates a particular exemplary embodiment describe herein. In particular, FIG. 4 is a flow chart of process 400 for accessing package 111 through data store 114. In operation, packager 112 receives (401) a request for package 111. In an exemplary embodiment, data seeker uses client device 106 to access data store 114 to view package 111. Data seeker selects package 111. Data store 114 is configured to generate a request for package 111, for example, following selection of package 111. Packager 112 verifies (402) the access level of data seeker. For example, if data seeker is requesting to view a package that is only accessible to subscribers and the data seeker is not a subscriber, then packager 112 denies the request of data seeker to access the package. In another example, if the package is associated with a one-time purchase access level, then the data seeker may access the data package assuming the data seeker pays the one-time purchase fee.

In the illustrative example of FIG. 4, packager 112 determines (404) if a transaction fee is associated with the requested package, for example, if the requested package is associated with a one-time purchase access level. If packager 112 determines that the requested package is associated with an access fee, packager 112 sends to client device 106 a request for the transaction fee. Packager 112 receives (408) the transaction fee. Following receipt of the transaction fee or if the requested package is not associated with a transaction fee, packager 112 sends (110) package 111 to client device 106 associated with data seeker.

Using the techniques described herein, medical data is collected and packaged for display in a data store. Data seekers may access the data store to view and to purchase the packages. Additionally, data seekers may post in the data store requests for data proposals for a particular type of data, for example, when the data seeker can not find a package including the particular type of data in the data store.

As used herein, the terms “computer” and “computer systems” refer broadly to any sort of combination of one or more servers and/or computing devices. As used herein, the terms “instrument(s)” and “medical study instrument(s)” refer broadly to any type of device and/or document (or any combination thereof), which presents data and/or information to a user and allows the user to input and/or send data and/or information to the system 102

Embodiments can be implemented in digital electronic circuitry, or in computer hardware, firmware, software, or in combinations thereof. An apparatus can be implemented in a computer program product tangibly embodied or stored in a machine-readable storage device for execution by a programmable processor; and method actions can be performed by a programmable processor executing a program of instructions to perform functions by operating on input data and generating output. The embodiments described herein, and other embodiments of the invention, can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language.

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Computer readable media for embodying computer program instructions and data include all forms of non-volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in special purpose logic circuitry. Any of the foregoing can be supplemented by, or incorporated in, ASICs (application-specific integrated circuits).

To provide for interaction with a user, embodiments can be implemented on a computer having a display device, e.g., a LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments can be implemented in a computing system that includes a back end component, e.g., as a data server, or that includes a middleware component, e.g., an application server, or that includes a front end component, e.g., a client computer having a graphical user interface or a Web browser through which a user can interact with an implementation of embodiments, or any combination of such back end, middleware, or front end components. The components of the system can be interconnected by any form or medium of digital data communication, e.g., a communication network. Examples of communication networks include a local area network (LAN) and a wide area network (WAN), e.g., the Internet.

The system and method or parts thereof may use the “World Wide Web” (Web or WWW), which is that collection of servers on the Internet that utilize the Hypertext Transfer Protocol (HTTP). HTTP is a known application protocol that provides users access to resources, which may be information in different formats such as text, graphics, images, sound, video, Hypertext Markup Language (HTML), as well as programs. Upon specification of a link by the user, the client computer makes a TCP/IP request to a Web server and receives information, which may be another Web page that is formatted according to HTML. Users can also access other pages on the same or other servers by following instructions on the screen, entering certain data, or clicking on selected icons. It should also be noted that any type of selection device known to those skilled in the art, such as check boxes, drop-down boxes, and the like, may be used for embodiments using web pages to allow a user to select options for a given component. Servers run on a variety of platforms, including UNIX machines, although other platforms, such as Windows 2000/2003, Windows NT, Sun, Linux, and Macintosh may also be used. Computer users can view information available on servers or networks on the Web through the use of browsing software, such as Firefox, Netscape Navigator, Microsoft Internet Explorer, or Mosaic browsers. The computing system can include clients and servers. A client and server are generally remote from each other and typically interact through a communication network. The relationship of client and server arises by virtue of computer programs running on the respective computers and having a client-server relationship to each other.

Other embodiments are within the scope and spirit of the description claims. In one embodiment, the rules described herein (e.g., the procedure determination rules or the medical assessment rules) are executed by a rules engine included in the system 102. In another embodiment, data collected by the system 102 through the instruments is stored in an EMR system 128. The research tool may then query the EMR system 128 for patient data matching one or more patient criteria. Through the network 112, the matching data is returned to the system 102 and the research tool processes and analyzes the returned data. In yet another embodiment, the techniques described herein are used to generate, review and validate instruments pertaining to various fields (e.g., the veterinary field, the legal field and the financial services field) and collect and retrieve data for the instruments pertaining to the various fields. In still another embodiment, the instrument generation module 116, the instrument validation module 118, the research tools module 120, the procedure determination module 122 and the patient flow module 124 are integrated together through various communication channels and/or are implemented as an instrument generation system, an instrument validation system, a research tools system, a procedure determination system and a patient flow system (collectively referred to as “the systems” herein, without limitation, for the purposes of convenience), with each system including one or more servers or computing devices and the systems being integrated together through various communication channels and/or network connections.

Additionally, due to the nature of software, functions described above can be implemented using software, hardware, firmware, hardwiring, or combinations of any of these. Features implementing functions may also be physically located at various positions, including being distributed such that portions of functions are implemented at different physical locations. The use of the term “a” herein and throughout the application is not used in a limiting manner and therefore is not meant to exclude a multiple meaning or a “one or more” meaning for the term “a.” Additionally, to the extent priority is claimed to a provisional patent application, it should be understood that the provisional patent application is not limiting but includes examples of how the techniques described herein may be implemented.

A number of exemplary embodiments of the invention have been described. Nevertheless, it will be understood by one of ordinary skill in the art that various modifications may be made without departing from the spirit and scope of the invention.

What is claimed is:

1. A computer-implemented method comprising:
receiving, by one or more computers, medical data that is a candidate for purchase in a data store;
sending the received medical data to a regulatory engine for review;
receiving, from the regulatory engine, approval to publish the medical data in the data store; and
publishing the medical data in the data store.

2. The computer-implemented method of claim 1, wherein the data store comprises a graphical user interface that when rendered on a display device renders a visual representation through which a consumer may view medical data.

3. The computer-implemented method of claim 1, further comprising:
receiving from a user a request to purchase medical data from the data store;
verifying that an access level of the user matches an access level associated with the medical data; and
sending to the user the purchased medical data.

4. The computer-implemented method of claim 1, further comprising:
verifying that the medical data may be formatted in accordance with one or more data format specifications; and
generating a package from the medical data, with the package comprising the medical data formatted according to the one or more data format specifications.

5. The computer-implemented method of claim 4, wherein publishing the medical data in the data store comprises:
generating a graphical user interface that when rendered in a display device renders a visual representation of at least some of the medical data included in the package.

6. The computer-implemented method of claim 3, further comprising:
receiving, by the one or more computers, a transaction fee for the requested medical data.

7. One or more machine-readable media configured to store instructions that are executable by one or more processing devices to perform operations comprising:
receiving medical data that is a candidate for purchase in a data store;
sending the received medical data to a regulatory engine for review;
receiving, from the regulatory engine, approval to publish the medical data in the data store; and
publishing the medical data in the data store.

8. The one or more machine-readable media of claim 7, wherein the data store comprises a graphical user interface that when rendered on a display device renders a visual representation through which a consumer may view medical data.

9. The one or more machine-readable media of claim 7, wherein the operations further comprise:
receiving from a user a request to purchase medical data from the data store;
verifying that an access level of the user matches an access level associated with the medical data; and
sending to the user the purchased medical data.

10. The one or more machine-readable media of claim 7, wherein the operations further comprise:
verifying that the medical data may be formatted in accordance with one or more data format specifications; and
generating a package from the medical data, with the package comprising the medical data formatted according to the one or more data format specifications.

11. The one or more machine-readable media of claim 10, wherein the operations further comprise:
generating a graphical user interface that when rendered in a display device renders a visual representation of at least some of the medical data included in the package.

12. The one or more machine-readable media of claim 9, wherein the operations further comprise:
receiving, by the one or more computers, a transaction fee for the requested medical data.

13. An electronic system comprising:
one or more processing devices; and
one or more machine-readable media configured to store instructions that are executable by the one or more processing devices to perform operations comprising:
receiving medical data that is a candidate for purchase in a data store;
sending the received medical data to a regulatory engine for review;
receiving, from the regulatory engine, approval to publish the medical data in the data store; and
publishing the medical data in the data store.

14. The electronic system of claim 13, wherein the data store comprises a graphical user interface that when rendered on a display device renders a visual representation through which a consumer may view medical data.

15. The electronic system of claim 13, wherein the operations further comprise:
receiving from a user a request to purchase medical data from the data store;
verifying that an access level of the user matches an access level associated with the medical data; and
sending to the user the purchased medical data.

16. The electronic system of claim 13, wherein the operations further comprise:
verifying that the medical data may be formatted in accordance with one or more data format specifications; and
generating a package from the medical data, with the package comprising the medical data formatted according to the one or more data format specifications.

17. The electronic system of claim 16, wherein the operations further comprise:
generating a graphical user interface that when rendered in a display device renders a visual representation of at least some of the medical data included in the package.

18. The electronic system of claim 15, wherein the operations further comprise:
receiving, by the one or more computers, a transaction fee for the requested medical data.
